(19) United States
(12) Patent Application Publication
(10) Pub. No.: US 2022/0313291 A1
(43) Pub. Date: Oct. 6, 2022

(54) PRIMING INSTRUMENT FOR MEDICAL DEVICE, MEDICAL DEVICE SYSTEM, AND METHOD FOR PRIMING MEDICAL DEVICE

(71) Applicant: TERUMO KABUSHIKI KAISHA, Tokyo (JP)

(72) Inventors: Taiga NAKANO, Irvine, CA (US); Bao DAN, Irvine, CA (US)

(73) Assignee: TERUMO KABUSHIKI KAISHA, Tokyo (JP)

(21) Appl. No.: 17/843,566

(22) Filed: Jun. 17, 2022

Related U.S. Application Data
(63) Continuation of application No. PCT/JP2020/013394, filed on Mar. 25, 2020.

(51) Int. Cl. A61B 17/22; A61B 17/3207

(52) U.S. Cl. CPC A61B 17/22; A61B 17/3207; A61B 2217/007; A61B 2017/22038; A61B 2017/22079

(57) ABSTRACT

A priming instrument for a medical device, a medical device system, and a method for priming a medical device, which are capable of priming the medical device having a plurality of lumens that open in a living body. The medical device includes a liquid delivering lumen having a first opening portion and a discharge lumen having a second opening portion. The priming instrument includes an instrument body having a hollow portion that covers the first opening portion and the second opening portion of the medical device, the hollow portion includes a distal portion and a proximal portion, the distal portion of the hollow portion is closed, the proximal portion of the hollow portion has an insertion port into which the medical device is insertable, and the insertion port includes a sealing portion that seals an inside of the hollow portion when the medical device is inserted into the hollow portion.

### CROSS-REFERENCES TO RELATED APPLICATIONS

This application is a continuation of International Application No. PCT/JP2020/013394 filed on Mar. 25, 2020, the entire content of which is incorporated herein by reference.

### TECHNOLOGICAL FIELD

The present disclosure relates to a priming instrument for a medical device having a plurality of lumens that open in a living body, a medical device system, and a method for priming a medical device.

### BACKGROUND DISCUSSION

Examples of treatment methods for a stenosed site caused by plaque and thrombus in a blood vessel include a method for dilating the blood vessel by using a balloon, and a method for causing a mesh-shaped or coil-shaped stent to indwell the blood vessel as a support for the blood vessel. However, it is difficult for these methods to treat a stenosed site that is hardened by calcification or a stenosed site that is formed at a bifurcated portion in the blood vessel.

Atherectomy devices are medical devices that can also perform treatment in such a case.

The atherectomy device is a device that removes the plaque in the blood vessel by shearing/breaking the plaque by a cutting portion that rotates at a relatively high speed. The atherectomy device includes the cutting portion disposed at a distal end of a catheter, a drive shaft that transmits the high-speed rotation of the cutting portion from outside a living body, and a tubular body that rotatably accommodates the drive shaft.

For example, an atherectomy device disclosed in U.S. Pat. No. 9,295,373 includes a liquid delivering lumen for supplying a fluid such as saline (or saline solution) to the vicinity of a distal end of a drive shaft, and an aspiration lumen for aspirating blood containing debris and the like cut by a cutting portion. As described above, among medical devices including the atherectomy device, a medical device having a plurality of lumens that open in a living body is known.

In order to prime the medical device having the liquid delivering lumen and the aspiration lumen before a procedure, a container is filled with saline, and a distal portion of the medical device is immersed in the container. Next, a liquid delivering pump and an aspiration pump of the medical device are operated to fill the liquid delivering lumen and the aspiration lumen with the saline. When the liquid delivering pump and the aspiration pump are stopped after a certain period of time elapses, the medical device is taken out from the container. In the case of a medical device that does not have an aspiration pump, the saline is aspirated into the aspiration lumen by, for example, rotating the drive shaft.

In the priming of the medical device having the plurality of lumens as described above, a plurality of pumps are required to be operated or a shaft is required to be rotated, which leads to complication of a priming procedure and complication of the medical device. In addition, the medical device may be damaged when a rotating portion is rotated before the lumens are filled with the fluid.

### SUMMARY

A priming instrument is disclosed for a medical device, a medical device system, and a method for priming a medical device, which are capable of rather easily priming the medical device having a plurality of lumens that open in a living body.

A priming instrument for a medical device is disclosed, which includes a liquid delivering lumen having a first opening portion and a discharge lumen having a second opening portion according to the present disclosure. The priming instrument for a medical device includes: an instrument body having a hollow portion that covers the first opening portion and the second opening portion of the medical device, in which the hollow portion includes a distal portion and a proximal portion, the distal portion of the hollow portion is closed, the proximal portion of the hollow portion has an insertion port into which the medical device is insertable, and the insertion port includes a sealing portion that seals an inside of the hollow portion in a state where the medical device is inserted into the hollow portion.

A medical device system according to the present disclosure includes: a medical device that includes a liquid delivering lumen having a first opening portion and a discharge lumen having a second opening portion; and the priming instrument.

A method for priming a medical device including a liquid delivering lumen having a first opening portion and a discharge lumen having a second opening portion according to the present disclosure includes: inserting a distal portion of the medical device from an insertion port of an instrument body and disposing the first opening portion and the second opening portion of the medical device in a hollow portion of the instrument body; sealing an inside of the hollow portion with a sealing portion provided in the insertion port of the instrument body; and injecting a fluid into the liquid delivering lumen from a proximal side, causing the fluid to flow into the hollow portion from the first opening portion of the liquid delivering lumen into which the fluid is injected, and causing the fluid to flow into the discharge lumen from the hollow portion via the second opening portion to fill the liquid delivering lumen and the discharge lumen with the fluid.

In the priming instrument for a medical device, the medical device system, and the method for priming a medical device configured as described above, each lumen can be filled with the fluid via the sealed hollow portion. At this time, since a rotating portion of the medical device is not required to be rotated and an aspiration is not required, the priming instrument for a medical device, the medical device system, and the method for priming a medical device can rather easily perform priming. In addition, the priming instrument for a medical device, the medical device system, and the method for priming a medical device can help prevent generation of air bubbles due to rotation of the rotating portion.

### BRIEF DESCRIPTION OF THE DRAWINGS

FIG. 1 is a plan view showing a medical device according to the present embodiment.

FIG. 2 is a diagram showing a casing of a handle of the medical device in a cross-sectional view, and other parts of the casing of the handle in a plan view.

FIG. 3 is a cross-sectional view showing a distal portion of the medical device.

FIG. 4 is a cross-sectional view showing a portion at a predetermined distance from a distal end to a proximal side of the medical device.

FIG. 5 is an enlarged cross-sectional view showing a part of the handle of the medical device.

FIG. 6 is a cross-sectional view showing a priming instrument.

FIG. 7 is a cross-sectional view showing a state in which the distal portion of the medical device is inserted into the priming instrument.

FIG. 8 is a cross-sectional view showing a state in which a sealing portion is sealed from the state shown in FIG. 7.

FIG. 9 is an enlarged cross-sectional view of the vicinity of a sealing portion of a priming instrument according to a first modification.

FIG. 10 is a cross-sectional view of a priming instrument according to a second modification.

FIG. 11 is a cross-sectional view of a priming instrument according to a third modification.

FIG. 12 is a cross-sectional view showing a state in which a distal portion of a medical device is inserted into the priming instrument according to the third modification.

FIG. 13 is a front view of a priming instrument according to a fourth modification.

FIGS. 14A-14C show schematic views of a state in which a lesion area is removed by a medical device, in which FIG. 14A shows a state in which cutting is started, FIG. 14B shows a state in which cutting is performed after an outer tube shaft is rotated, and FIG. 14C shows a state in which cutting is performed while the outer tube shaft is moved.

### DETAILED DESCRIPTION

Set forth below with reference to the accompanying drawings is a detailed description of embodiments of a priming instrument for a medical device having a plurality of lumens that open in a living body, a medical device system, and a method for priming a medical device. Note that since embodiments described below are preferred specific examples of the present disclosure, although various technically preferable limitations are given, the scope of the present disclosure is not limited to the embodiments unless otherwise specified in the following descriptions. It is noted that a size and a ratio of each member in the drawings may be exaggerated for convenience of description and may differ from an actual size and ratio. In addition, in the present specification, a side of a medical device to be inserted into a body lumen is referred to as a “distal side”, and a side to be operated is referred to as a “proximal side”.

A medical device 10 according to the present embodiment can be inserted into a blood vessel in an acute lower limb ischemia or a deep vein thrombosis, and can be used for a procedure for destroying and removing a thrombus, a plaque, an atheroma, a calcified lesion, and the like. It is noted that an object to be removed is not necessarily limited to the thrombus, the plaque, the atheroma, and the calcified lesion, and any object that may be present in a body lumen or a body cavity may be applicable.

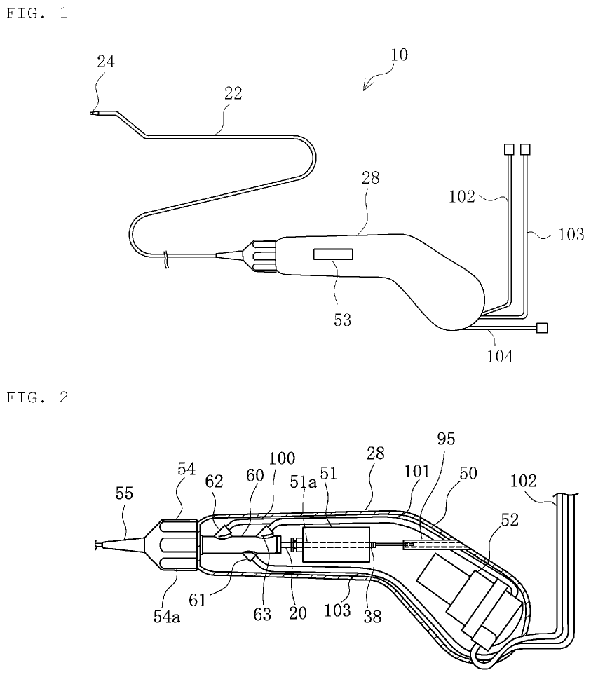

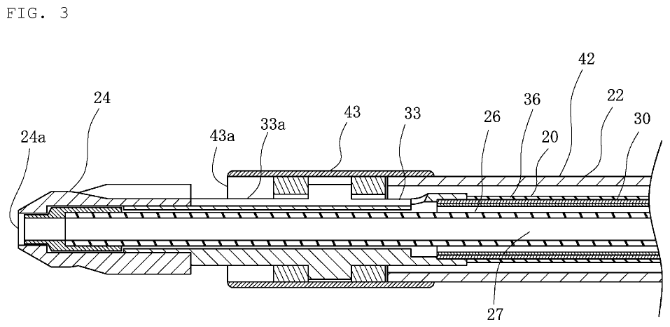

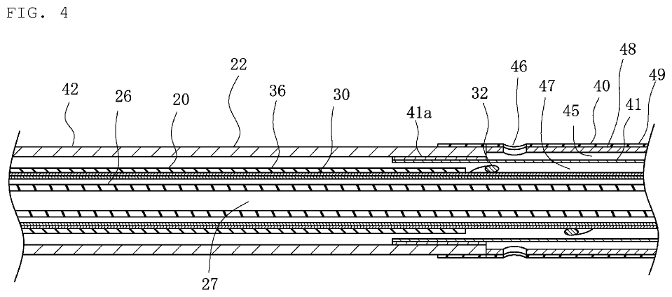

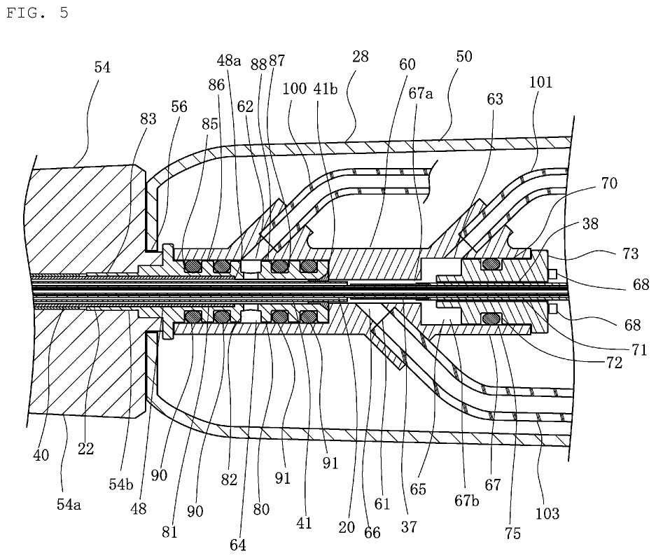

As shown in FIGS. 1 to 4, the medical device 10 includes a long drive shaft 20 that is rotationally driven, an outer tube shaft 22 that accommodates the drive shaft 20, a cutting portion 24 that cuts a thrombus, a guide wire lumen tube 26 that is disposed inside the drive shaft 20, and a handle portion 28.

As shown in FIGS. 2 to 4, the drive shaft 20 is an elongated tubular body that transmits a rotational force to the cutting portion 24. The drive shaft 20 includes a coil member 30, a conveying coil 32 that generates a conveying force, and a rotating shaft portion 33 that is rotatably supported by a distal bearing portion 43, which will be described later, of the outer tube shaft 22. The drive shaft 20 further includes a first protective tube 36 disposed on an outer periphery of a distal portion of the drive shaft 20, a second protective tube 37 disposed on an outer periphery of a proximal portion of the drive shaft 20, and a proximal tube 38 disposed on an outer periphery of the drive shaft 20 on the proximal side with respect to the second protective tube 37.

As a constituent material for the coil member 30, for example, stainless steel, Ta, Ti, Pt, Au, W, polyolefins such as polyethylene and polypropylene, polyamides, polyesters such as polyethylene terephthalate, fluoropolymers such as an ethylene tetrafluoroethylene copolymer (ETFE), polyether ether ketone (PEEK), and polyimides can be used.

The guide wire lumen 26 is a tubular body disposed inside the drive shaft 20. The guide wire lumen 26 has a guide wire lumen 27 through which a guide wire W passes. The guide wire lumen 26 helps prevent the guide wire W passing through the guide wire lumen 27 from rubbing against the drive shaft 20. A distal portion of the guide wire lumen 26 protrudes toward the distal side with respect to the drive shaft 20, and is disposed inside the cutting portion 24. A proximal portion of the guide wire lumen 26 is interlocked with a hub proximal tube 95 disposed in the handle portion 28.

The outer tube shaft 22 is an elongated tubular body that accommodates the drive shaft 20. The outer tube shaft 22 includes an outer layer 40, an inner layer 41, a shaping shaft 42, the distal bearing portion 43 disposed on the distal side with respect to the shaping shaft 42, and a seal holding portion 80 to which proximal portions of the inner layer 41 and the outer layer 40 are fixed. A liquid delivering lumen 45 for delivering a liquid such as saline (or saline solution) to the distal side is formed between the outer layer 40 and the inner layer 41. At least one liquid delivering opening portion 46 penetrating from an inner peripheral surface to an outer peripheral surface is formed in a distal portion of the outer layer 40. A discharge lumen 47 for discharging the object such as a cut thrombus in a proximal direction is formed inside the inner layer 41.

The outer layer 40 includes a torque shaft 48 and an outer sheath 49 that is in close contact with an outer peripheral surface of the torque shaft 48.

The torque shaft 48 is a tubular body that transmits a torque applied by an operator on the handle portion 28 to the distal side. A proximal portion of the torque shaft 48 is fixed to an inner peripheral surface of the seal holding portion 80. A distal portion of the torque shaft 48 is fixed to a proximal portion of the shaping shaft 42. The aforementioned liquid delivering lumen 45 is formed inside the torque shaft 48. It is preferable that the torque shaft 48 has flexibility so as to be bent in the body lumen and has relatively high torque transmission performance. As a constituent material for the torque shaft 48, for example, a circular tube formed of a metal material having a certain degree of strength, in which spiral slits or grooves are formed by laser processing, can be used. As to the metal material, for example, stainless steel, Ta, Ti, Pt, Au, or W can be used.

The outer sheath 49 is a tubular body that is in contact with the outer peripheral surface of the torque shaft 48. The outer sheath 49 helps prevent leakage of the liquid in the liquid delivering lumen 45 from a gap of the spiral slits formed in the torque shaft 48. The outer sheath 49 can be formed, for example, of a heat-shrinkable tube whose diameter is reduced by heating and which is brought into contact with the torque shaft 48.

The inner layer 41 is disposed inside the outer layer 40 with a gap between the inner layer 41 and the outer layer 40. The gap between the inner layer 41 and the outer layer 40 can be the liquid delivering lumen 45. The discharge lumen 47 for discharging the object such as a cut thrombus in the proximal direction is formed inside the inner layer 41. The inner layer 41 can be, for example, a tubular body in which a braided wire braided in a tubular shape is embedded in a resin material as a reinforcement body. A distal portion of the inner layer 41 is fixed to an inner peripheral surface of the shaping shaft 42 with a first sealing portion 41a which can be, for example, an adhesive or the like. The proximal portion of the inner layer 41 protrudes toward the proximal side with respect to the outer layer 40, and is fixed to the inner peripheral surface of the seal holding portion 80 with a second sealing portion 41b which can be, for example, an adhesive or the like.

The resin material forming the inner layer 41 preferably has a certain degree of flexibility and low friction, and polyether ether ketone (PEEK), fluoropolymers such as PTFE and ETFE, polymethyl methacrylate (PMMA), polyethylene (PE), a polyether block amide copolymer (PEBAX), polyimides, or a combination of polyether ether ketone (PEEK), fluoropolymers such as PTFE and ETFE, polymethyl methacrylate (PMMA), polyethylene (PE), a polyether block amide copolymer (PEBAX), and polyimides can be used.

The shaping shaft 42 is a tubular body to which the distal portion of the torque shaft 48 is fixed. The shaping shaft 42 is bent at two positions such that an axis of the proximal portion and an axis of a distal portion of the shaping shaft 42 are shifted from each other. It is noted that the number of the bent portions of the shaping shaft 42 may be one (1) or three (3) or more. The shaping shaft 42 can be used for changing positions and orientations of a distal portion of the outer tube shaft 22 and the cutting portion 24 by rotating.

The distal bearing portion 43 is disposed at the most distal end of the outer tube shaft 22, and rotatably supports the rotating shaft portion 33 provided on the drive shaft 20. The distal bearing portion 43 is fixed to the distal portion of the shaping shaft 42. On the distal side of the distal bearing portion 43, a distal opening portion 43a is formed which aspirates the object such as a cut thrombus or the liquid discharged from the liquid delivering opening portion 46 and takes the object and the liquid into the discharge lumen 47. A distal end of the distal bearing portion 43 is located on the proximal side of the cutting portion 24.

The proximal portion of the outer layer 40 and the proximal portion of the inner layer 41 are fixed to the seal holding portion 80, and at least a part of the seal holding portion 80 is disposed inside a housing 60 provided in the handle portion 28. The seal holding portion 80 can hold a plurality of seals and is rotatably supported inside the housing 60. The seal holding portion 80 includes a holding portion lumen 81 penetrating along an axis of the drive shaft 20, a supply port 82 penetrating from an outer peripheral surface of seal holding portion 80 to the holding portion lumen 81, and an operation fixing portion 83 fixed to an operation portion 54 to be described later. The seal holding portion 80 further includes two first recesses 85 formed on the outer peripheral surface of the seal holding portion 80 on the distal side with respect to the supply port 82, three first stoppers 86 arranged alternately with the first recesses 85, two second recesses 87 formed on the outer peripheral surface of the seal holding portion 80 on the proximal side with respect to the supply port 82, and three second stoppers 86 alternately arranged with the second recesses 87.

At least a part of the outer tube shaft 22, the drive shaft 20, and the guide wire lumen tube 26 pass through the holding portion lumen 81. The torque shaft 48 of the outer layer 40 is fixed to the distal side of the holding portion lumen 81 with respect to the supply port 82, for example, with an adhesive or the like. The inner layer 41 protruding inside the torque shaft 48 from a proximal opening portion 48a of the torque shaft 48 in the proximal direction is fixed to the proximal side of the holding portion lumen 81 with respect to the supply port 82 with the second sealing portion 41b. Therefore, the liquid delivering lumen 45 between the outer layer 40 and the inner layer 41 communicates with the supply port 82.

Each of the first recesses 85 accommodates a first elastic body 90 such as an O-ring. The first elastic bodies 90 maintain liquid-tightness between the seal holding portion 80 and the housing 60 while maintaining a state in which the seal holding portion 80 is rotatable inside the housing 60. The first stoppers 86 helps prevent the first elastic bodies 90 from falling off the first recesses 85. Each of the second recesses 87 can accommodate a second elastic body 91 such as an O-ring. The second elastic bodies 91 help maintain the liquid-tightness between the seal holding portion 80 and the housing 60 while maintaining the state in which the seal holding portion 80 is rotatable inside the housing 60. The second stoppers 86 helps prevent the second elastic bodies 91 from falling off the second recesses 87.

An anti-kink protector 55 and the operation portion 54 can be fixed to an outer peripheral surface of a proximal portion of the outer tube shaft 22. The anti-kink protector 55 helps prevent kink at the proximal portion of the outer tube shaft 22. An outer surface of the operation portion 54 may be formed with irregularities so as to be easily caught or grasped by fingers of the operator.

Constituent materials for the torque shaft 48 and the shaping shaft 42 preferably have a certain degree of strength, and for example, stainless steel, Ta, Ti, Pt, Au, W, and a shape memory alloy can be used. A constituent material for the outer tube shaft 22 may be a resin, for example, an engineering plastic such as an ABS resin, polycarbonate (PC), polymethyl methacrylate (PMMA), polyacetal (POM), polyphenyl sulfone (PPSU), polyethylene (PE), a carbon fiber, or polyether ether ketone (PEEK).

The cutting portion 24 is a member that cuts the object such as a thrombus. The cutting portion 24 is fixed to an outer peripheral surface of the distal portion of the drive shaft 20. The cutting portion 24 can have a large number of minute abrasive grains on a surface of the cutting portion. Alternatively, the cutting portion 24 may include a sharp blade. The cutting portion 24 has an opening portion 24a that opens to the distal side. The opening portion 24a communicates with a distal end of the guide wire lumen 27, and is a portion through which the guide wire lumen 27 communicates with an outside on the distal side.

The rotating shaft portion 33 is rotatably supported by the distal bearing portion 43 provided on the outer tube shaft 22. A proximal portion of the rotating shaft portion 33 is fixed to the coil member 30, and a distal portion of the rotating shaft portion 33 is fixed to the cutting portion 24. The rotating shaft portion 33 has at least one groove-shaped passage 33a extending along the axis. The passage 33a allows the object cut by the cutting portion 24 to pass through an inside of the distal bearing portion 43 in the proximal direction.

A constituent material for the cutting portion 24 preferably has sufficient strength to cut a thrombus, and for example, stainless steel, Ta, Ti, Pt, Au, W, a shape memory alloy, and a cemented carbide can be used.

The handle portion 28 can include a casing 50, a drive unit 51, a liquid delivering unit 52, a switch 53, a suction tube 102, a first liquid delivering tube 100, a second liquid delivering tube 101, a discharge tube 103, and an electric cable 104. The handle portion 28 can further include the operation portion 54, the housing 60, and a shaft support 70.

The casing 50 forms an outline of the handle portion 28. The casing 50 accommodates the drive unit 51, the housing 60, the shaft support 70, the first liquid delivering tube 100, the second liquid delivering tube 101, a part of the discharge tube 103, and a part of the electric cable 104. A passage hole 56 through which the drive shaft 20, the outer tube shaft 22, and the guide wire lumen tube 26 pass is formed in a distal portion of the casing 50.

The drive unit 51 can be, for example, a hollow motor. The drive unit 51 can include a hollow power shaft 51a that generates a driving force by an electric power supplied from an outside via the electric cable 104. The power shaft 51a passes through the drive unit 51 and rotates while being supported by a bearing. The drive shaft 20 is accommodated in the power shaft 51a. An inner peripheral surface of the power shaft 51a is in slidable contact with an outer peripheral surface of the drive shaft 20. The drive shaft 20 substantially penetrates the power shaft 51a, and the proximal portion of the drive shaft 20 is fixed to a proximal portion of the power shaft 51a, for example, by welding, adhesion, or the like. A rotation speed of the power shaft 51a is not particularly limited, and can be, for example, 5,000 rpm (revolutions per minute) to 200,000 rpm. The drive unit 51 can be connected to a control apparatus, and can be controlled from an inside or an outside of the handle portion 28.

The electric cable 104 can be connected to an external power supply or the control apparatus. The switch 53 is a portion operated by the operator to drive and stop the drive unit 51. The switch 53 is located on an outer surface of the casing 50. It is noted that when a battery is provided in the handle portion 28, an electric cable is located in the handle portion 28 and connected to the battery. When the electric cable 104 is connected to the external power supply, the control apparatus can be provided in the handle portion 28 to perform signal processing on an operation input of the switch 53 and control the drive unit 51 and the liquid delivering unit 52.

The operation portion 54 is a portion that is operated by the operator with the fingers to apply a rotational torque to the outer tube shaft 22. The operation portion 54 is fixed to the operation fixing portion 83 of the seal holding portion 80 to which the outer tube shaft 22 is fixed. The operation portion 54 includes an operation dial 54a and a fixing recess 54b. The operation dial 54a is a substantially disk-shaped portion operated by the operator with the fingers. An outer peripheral surface of the operation dial 54a has relatively high frictional resistance so as to be rather easily operated. The operation fixing portion 83 of the seal holding portion 80 is fitted into the fixing recess 54b so as to be fixed. When the operator rotates the operation portion 54, the outer tube shaft 22 fixed to the operation portion 54 rotates with respect to the drive shaft 20, the guide wire lumen tube 26, and the casing 50. At this time, the first elastic bodies 90 and the second elastic bodies 91 held by the seal holding portion 80 to which the operation portion 54 is fixed slide on the outer peripheral surface of the seal holding portion 80 while receiving frictional resistance. The frictional resistance of the first elastic bodies 90 and the second elastic bodies 91 with respect to the seal holding portion 80 is large enough to hold an orientation of a rotation direction of the outer tube shaft 22. Therefore, when the operator releases the fingers after rotating the operation dial 54a, a rotated position can be held by the frictional resistance of the first elastic bodies 90 and the second elastic bodies 91 with respect to the seal holding portion 80. It is noted that the first elastic bodies 90 and the second elastic bodies 91 may be held on the outer peripheral surface of the seal holding portion 80 and slide on an inner peripheral surface of the housing 60.

The housing 60 includes a discharge port 61 through which the liquid or the object is discharged, and a first liquid delivering port 62 and a second liquid delivering port 63 through which the liquid is delivered. The housing 60 further includes a first liquid delivering lumen 64 with which the first liquid delivering port 62 communicates, a second liquid delivering lumen 65 with which the second liquid delivering port 63 communicates, and a discharge lumen 66 with which the discharge port 61 communicates.

The first liquid delivering lumen 64 has a predetermined inner diameter and is disposed in a distal portion of the housing 60. The first liquid delivering lumen 64 rotatably accommodates the seal holding portion 80. The first elastic bodies 90 and the second elastic bodies 91 held by the seal holding portion 80 are in slidable contact with an inner peripheral surface of the first liquid delivering lumen 64. The first liquid delivering port 62 is formed at a position communicating with the supply port 82 of the seal holding portion 80. The first liquid delivering port 62 is located on the proximal side with respect to the first elastic bodies 90, and is located on the distal side with respect to the second elastic bodies 91. The first liquid delivering port 62 is connected to the first liquid delivering tube 100 and can receive the liquid from the first liquid delivering tube 100. The liquid delivered from the first liquid delivering port 62 can flow from the supply port 82 into the liquid delivering lumen 45 formed between the torque shaft 48 and the inner layer 41. At this time, the first elastic bodies 90 helps prevent the liquid in the first liquid delivering lumen 64 from leaking to an outside of the housing 60. In addition, the second elastic bodies 91 helps prevent the liquid in the second liquid delivering lumen 65 from leaking to the discharge lumen 66. In addition, the second sealing portion 41b that fixes the inner layer 41 and the seal holding portion 80 helps prevent the liquid flowing into the liquid delivering lumen 45 from leaking to the discharge lumen 66. It is noted that the first elastic bodies 90 and the second elastic bodies 91 may be held by a groove portion formed on the inner peripheral surface of the first liquid delivering lumen 64 instead of the seal holding portion 80.

The second liquid delivering lumen 65 has a predetermined inner diameter and is disposed in a proximal portion of the housing 60. The housing 60 includes an accommodation portion 67 that accommodates the shaft support 70 that seals the second liquid delivering lumen 65. The accommodation portion 67 includes a first accommodation portion 67a that accommodates the proximal portion of the drive shaft 20, and a second accommodation portion 67b that has an inner diameter larger than that of the first accommodation portion 67a and accommodates the shaft support 70. A third elastic body 75 held by the shaft support 70 is in contact with an inner peripheral surface of the second liquid delivering lumen 65. A position of the second liquid delivering lumen 65 which is in contact with the third elastic body 75 is on the proximal side with respect to the second liquid delivering port 63. A distal side of the second liquid delivering lumen 65 communicates with the discharge lumen 66. The second liquid delivering port 63 is connected to the second liquid delivering tube 101 and can receive the liquid from the second liquid delivering tube 101. The liquid delivered from the second liquid delivering port 63 can flow into the discharge lumen 66 communicating with the second liquid delivering lumen 65. At this time, the third elastic body 75 helps prevents the liquid in the second liquid delivering lumen 65 from leaking to the outside of the housing 60.

The discharge lumen 66 is disposed between the first liquid delivering lumen 64 and the second liquid delivering lumen 65. The discharge lumen 66 communicates with the second liquid delivering lumen 65. An inner diameter of the discharge lumen 66 is smaller than the inner diameter of the first liquid delivering lumen 64 and also smaller than the inner diameter of the second liquid delivering lumen 65. The discharge port 61 is connected to the discharge tube 103. A proximal end of the outer tube shaft 22 is open in the discharge lumen 66. The drive shaft 20 protruding from the proximal end of the outer tube shaft 22 in the proximal direction passes through the discharge lumen 66 and further extends in the proximal direction. Therefore, the discharge lumen 47 formed between the outer tube shaft 22 and the drive shaft 20 communicates with the discharge lumen 66. The discharge lumen 66 can receive the liquid or the object from the discharge lumen 47, and the liquid or the object can be discharged from the discharge port 61 to the discharge tube 103.

The shaft support 70 is a member that is inserted into the second accommodation portion 67b of the accommodation portion 67 from a proximal side of the housing 60, holds the distal side with respect to the drive unit 51, that is, the proximal portion of the drive shaft 20, and partially closes the second liquid delivering lumen 65. The shaft support 70 includes a support lumen 71 penetrating along the axis of the drive shaft 20, a third recess 72 formed in an outer peripheral surface of the shaft support 70, and a lid portion 73 having an outer diameter larger than the inner diameter of the second accommodation portion 67b and facing a proximal surface of the housing 60. The drive shaft 20 and the guide wire lumen 26 pass through the support lumen 71.

The third recess 72 accommodates the third elastic body 75, for example, such as an O-ring. The third elastic body 75 is in contact with an outer peripheral surface of the shaft support 70 and an inner peripheral surface of the second accommodation portion 67b, and maintains liquid-tightness between the shaft support 70 and the second liquid delivering lumen 65. The third recess 72 has a width larger than that of the third elastic body 75.

A distal surface of the lid portion 73 faces a proximal surface of the second accommodation portion 67b. A proximal surface of the lid portion 73 faces stopper portions 68 protruding from the housing 60. Movement of the shaft support 70 in an axial direction is restricted by the stopper portions 68, and detachment of the shaft support 70 from the housing 60 can be prevented.

Examples of constituent materials for the first elastic bodies 90, the second elastic bodies 91, and the third elastic body 75 can include silicone rubber, ethylene propylene rubber, nitrile rubber, chloroprene rubber, isoprene rubber, butyl rubber, styrene butadiene rubber, natural rubber such as polyurethane, synthetic rubber, and a silicone resin. It is noted that as the constituent materials for the first elastic bodies 90, the second elastic bodies 91, and the third elastic body 75, a resin material such as PTFE, FEP, or nylon may be used. As shown in FIG. 6, cross-sectional shapes of the first elastic bodies 90, the second elastic bodies 91, and the third elastic body 75 are not limited to a circular or elliptical shape, and may be, for example, a rectangular shape.

Examples of constituent materials for the housing 60, the seal holding portion 80, the lid portion 73, and the operation portion 54 include ultra-high molecular weight polyethylene, polyester, polyamides, fluorine-based resins such as polytetrafluoroethylene, an ABS resin, polyacetal (POM), polycarbonate (PC), polypropylene (PP), polybutylene terephthalate (PBT), polymethyl methacrylate (PMMA), and a combination of two or more of the polymer alloys, polymer blends, laminates, or the like.

The liquid delivering unit 52 is a pump that delivers the liquid to the housing 60 via the first liquid delivering tube 100 and the second liquid delivering tube 101. The liquid delivering unit 52 is connected to the suction tube 102 that receives a supply of the liquid such as saline from a liquid delivering source outside the housing 60, and can suction the liquid from the suction tube 102. The liquid delivering unit 52 is connected to the first liquid delivering tube 100 and the second liquid delivering tube 101, and can discharge the suctioned liquid to the first liquid delivering tube 100 and the second liquid delivering tube 101. The external liquid delivering source can be, for example, a saline bag, but is not limited to a saline bag. The liquid delivering unit 52 may be provided outside the handle portion 28 instead of being provided in the handle portion 28. The liquid delivering unit 52 is not limited to a pump as long as a liquid delivering pressure can be generated, and may be, for example, a syringe, a bag suspended from a drip tower, or a pressurized bag.

The discharge tube 103 is a tube that discharges the liquid or the object to the outside of the housing 60. The discharge tube 103 is connected to, for example, a waste liquid bag capable of accommodating the liquid or the object. It is noted that the discharge tube 103 may be connected to an aspiration source that can perform active aspiration, such as a pump or a syringe.

It is noted that the proximal portion of the drive shaft 20 is connected to the power shaft 51a via a proximal shaft 38 provided on an outer peripheral side of the drive shaft 20. The drive shaft 20 can be, for example, welded or bonded at a proximal portion of the proximal shaft 38, and the proximal shaft 38 can be bonded or welded to the power shaft 51a, whereby the drive shaft 20 is fixed. When the shaft support 70 and the drive shaft 20 are directly connected to each other, a large amount of leakage occurs due to the rotation of the drive shaft 20 formed with the coil member 30, but the leakage can be reduced by interposing the proximal shaft 38 between the shaft support 70 and the drive shaft 20.

Next, a priming instrument 200 for the medical device 10 will be described. The priming instrument 200 is used for filling the plurality of lumens of the medical device 10 with a liquid before the medical device 10 is used. As shown in FIG. 6, the priming instrument 200 includes an instrument body 210 having a hollow portion 212 in the instrument body 210, and a sealing portion 220 that seals the instrument body 210.

The instrument body 210 is formed in an elongated shape along an axial direction, and the hollow portion 212 inside the instrument body 210 is also elongated along the axial direction. A distal portion 212a of the hollow portion 212 is closed, and a proximal portion 212b of the hollow portion 212 has an insertion port 214 into which the medical device 10 is insertable. A distal portion of the medical device 10 inserted from the insertion port 214 can be accommodated in the hollow portion 212. The hollow portion 212 has an inner diameter and a length capable of covering from a distal end of the medical device 10 to a proximal side with respect to the liquid delivering opening portion (first opening portion) 46. Therefore, the liquid delivering opening portion 46, the distal opening portion (second opening portion) 43a, and the opening portion (third opening portion) 24a of the guide wire lumen 27 of the medical device 10 can be disposed in the hollow portion 212. The distal portion 212a of the hollow portion 212 has a concave curved surface shape. An inner surface of the instrument body 210 is preferably coated with a hydrophilic coating.

The sealing portion 220 is attached to the insertion port 214. The sealing portion 220 includes a base member 222 fixed to the instrument body 210, a seal member 221 accommodated in the base member 222, and a cap member 223 rotatably attached to the base member 222.

It is desirable that the instrument body 210 is partially or entirely formed of a flexible material. In a case where a part of the instrument body 210 is formed of the flexible material, it is desirable that a distal side of the instrument body 210 is formed of the flexible material. In addition, it is desirable that the instrument body 210 is formed of a transparent or translucent material. The material that constituents the instrument body 210, can be, for example, polyethylene terephthalate (PET), vinyl chloride (PVC), polycarbonate (PC), polymethyl methacrylate (PMMA), a fluorine-based resin, a silicone resin, or the like.

The base member 222 has a base lumen 240 communicating with the hollow portion 212. The base lumen 240 has an inner diameter equal to that of the hollow portion 212, and the medical device 10 can be inserted through the base lumen 240. The base member 222 includes a fixing portion 241 fitted to the instrument body 210, and a hollow accommodation recess 242 for accommodating the seal member 221. The accommodation recess 242 is disposed on a proximal side of the base lumen 240, and is formed to have an inner diameter larger than that of the base lumen 240 via a step portion 242a. In addition, the base member 222 includes an outer peripheral screw portion 243 that screws the cap member 223 on an outer peripheral surface of a proximal side. A stopper portion 244 protruding toward an outer peripheral side is provided at a distal portion of the outer peripheral screw portion 243.

The seal member 221 can be a tubular member having a seal lumen 230 having an inner diameter larger than an outer diameter of the thickest portion (i.e., portion having the greatest width or diameter) of the distal portion of the medical device 10. Since the seal member 221 is formed of an elastic material, the inner diameter of the seal lumen 230 can be reduced by being compressed in an axial direction. The seal member 221 is accommodated in the accommodation recess 242 of the base member 222, and a distal surface 232 is attached to the step portion 242a of the accommodation recess 242.

Examples of a material for forming the seal member 221 can include various rubbers such as silicone rubber, fluororubber, isoprene, and natural rubber, various resins such as polyurethane, a polyamide elastomer, polybutadiene, and soft vinyl chloride, and a combination of two or more of silicone rubber, fluororubber, isoprene, and natural rubber, various resins such as polyurethane, a polyamide elastomer, polybutadiene, and soft vinyl chloride. As a material for forming the seal member 221, silicone rubber is particularly preferable from the viewpoints of relatively small changes in physical properties, high dimensional accuracy during molding, and high durability for retaining the inner diameter of the seal member 221.

The cap member 223 has a cap lumen 250 communicating with the seal lumen 230. The cap lumen 250 has an inner diameter equal to or slightly larger than that of the seal lumen 230, and the medical device 10 can be inserted through the cap lumen 250. A distal surface of the cap member 223 can be a compression surface portion 252 that faces or is attached to a proximal surface 231 of the seal member 221.

An outer peripheral surface of the cap member 223 includes a cylindrical operation portion 251 that is manually rotated by the operator. An inner peripheral screw portion 253 to be screwed with the outer peripheral screw portion 243 of the base member 222 is formed on an inner peripheral side of the operation portion 251. Since the inner peripheral screw portion 253 is screwed with the outer peripheral screw portion 243 of the base lumen 240, the cap member 223 can be moved in the axial direction with respect to the base member 222 by rotating the cap member 223.

Next, a method of using the priming instrument 200 will be described together with an operation. First, in a state in which the cap member 223 of the priming instrument 200 is loosened, that is, in a state in which the cap member 223 is located on the proximal side with respect to the base member 222, the medical device 10 is inserted into the hollow portion 212 as shown in FIG. 7. In the state in which the cap member 223 is loosened, the seal lumen 230 has the inner diameter larger than an outer diameter of the medical device 10. Therefore, the medical device 10 can enter an inside of the hollow portion 212 from the insertion port 214 through the cap lumen 250, the seal lumen 230, and the base lumen 240. The medical device 10 is inserted until the distal end reaches the vicinity of the distal portion 212a of the hollow portion 212.

Since the distal portion 212a of the hollow portion 212 has the concave curved surface shape, the opening portion 24a of the medical device 10 can be prevented from being sealed with a distal surface of the hollow portion 212. In addition, the shape of the distal portion 212a can help prevent a distal portion of the cutting portion 24 disposed at the distal end of the medical device 10 from being damaged.

Since the instrument body 210 is formed of the flexible material, it may be difficult to damage the medical device 10. In addition, the instrument body 210 can be deformed in accordance with a shape of the distal portion of the medical device 10 which is shaped, and the damage to the medical device 10 inserted into the hollow portion 212 can be further prevented.

Since the instrument body 210 is formed of the transparent or translucent material, the operator can visually recognize an insertion position of the medical device 10. Therefore, the medical device 10 can be inserted to an appropriate position and can be prevented from being damaged.

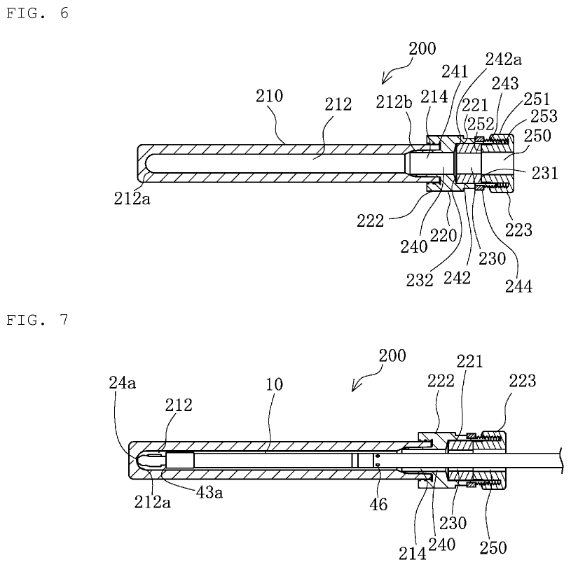

In the medical device 10 inserted into the hollow portion 212, the opening portion 24a communicating with the guide wire lumen 27, the distal opening portion 43a communicating with the discharge lumen 47, and the liquid delivering opening portion 46 communicating with the liquid delivering lumen 45 are all disposed in the hollow portion 212. In this state, the operator turns the cap member 223 to move the cap member 223 to a distal side. As shown in FIG. 8, when the cap member 223 is moved to the distal side, the compression surface portion 252, which is the distal surface of the cap member 223, presses the seal member 221 from the proximal surface 231 along the axial direction. Accordingly, the seal member 221 is sandwiched between the compression surface portion 252 and the step portion 242a and compressed in the axial direction, and since the inner diameter of the seal lumen 230 is reduced, the seal member 221 is brought into pressure contact with the outer tube shaft 22 of the medical device 10. Accordingly, the inside of the hollow portion 212 is sealed in a liquid-tight manner.

When the cap member 223 is rotated with respect to the base member 222 and moved toward the distal side, as shown in FIG. 8, a distal surface of the operation portion 251 is attached to the stopper portion 244. When the cap member 223 is attached to the stopper portion 244, the cap member 223 cannot further move toward the distal side. That is, the stopper portion 244 restricts the cap member 223 from moving by a predetermined distance or more in the axial direction. Accordingly, it is possible to prevent the seal member 221 from being excessively compressed due to excessive rotation of the cap member 223. Since the seal member 221 is not excessively compressed, the damage to the medical device 10 can be prevented. By setting a state in which the cap member 223 is attached to the stopper portion 244 to provide an optimum sealing force, a necessary and sufficient sealing force can be stably provided even if the operator does not consider about the force.

After the hollow portion 212 is sealed, the operator operates the switch 53 of the handle portion 28 to start an operation of the liquid delivering unit 52. Accordingly, the saline is suctioned from the suction tube 102 into the liquid delivering unit 52 and is discharged to the first liquid delivering tube 100 and the second liquid delivering tube 101. The saline discharged to the first liquid delivering tube 100 flows into the first liquid delivering lumen 64 of the housing 60 from the first liquid delivering port 62. The saline flowing into the first liquid delivering lumen 64 from the first liquid delivering port 62 flows into the liquid delivering lumen 45 formed between the outer layer 40 and the inner layer 41 from the supply port 82 of the seal holding portion 80. The saline entering the liquid delivering lumen 45 moves in a distal direction. The saline moved in the liquid delivering lumen 45 in the distal direction is discharged into the hollow portion 212 from the liquid delivering opening portion 46 formed in the distal portion of the outer layer 40.

As the liquid delivering unit 52 continues to operate, the inside of the hollow portion 212 sealed with the sealing portion 220 is filled with the saline discharged from the liquid delivering lumen 45. When the inside of the hollow portion 212 is filled with the saline, the saline flows into the guide wire lumen 27 from the opening portion 24a. In addition, the saline flows into the discharge lumen 47 from the distal opening portion 43a. When an internal pressure of the hollow portion 212 rises due to the flowing saline, the saline flows through the guide wire lumen 27 and the discharge lumen 47 from the distal side toward the proximal side, respectively. Accordingly, the guide wire lumen 27 and the discharge lumen 47 are also filled with the saline.

When a predetermined time elapses after the operator operates the switch 53 of the handle portion 28, the liquid delivering unit 52 is stopped. After the liquid delivering unit 52 is stopped, the operator loosens the cap member 223 of the priming instrument 200, expands the inner diameter of the seal member 221, and releases the sealing of the hollow portion 212. Then, the operator takes out the distal portion of the medical device 10 from the hollow portion 212. The stopping operation of the liquid delivering unit 52 can be performed by software of the control apparatus or by the switch operation of the operator.

In this manner, the distal portion of the medical device 10 is inserted into the hollow portion 212 of the priming instrument 200, the hollow portion 212 is sealed in a state in which the opening portions communicating with the plurality of lumens are disposed in the hollow portion 212, and the liquid is delivered by using one lumen to flow into the hollow portion 212, whereby all the lumens can be filled with the fluid. In this case, it is not necessary to rotate the drive shaft 20 and the cutting portion 24, and it is not necessary to aspirate the fluid. Therefore, since the drive shaft 20 and the cutting portion are not required to rotate in a state in which the lumens are not filled with the fluid, the medical device 10 can be prevented from being damaged at the time of priming. In addition, generation of air bubbles due to the rotation of the cutting portion 24 can also be prevented. When the inner surface of the instrument body 210 is coated with the hydrophilic coating, the generation of the air bubbles can be further prevented.

It is noted that when the medical device 10 is inserted into a living body after priming, removed, and then re-inserted into the living body, the priming is performed again using the priming instrument 200 immediately after the medical device 10 is removed from the living body.

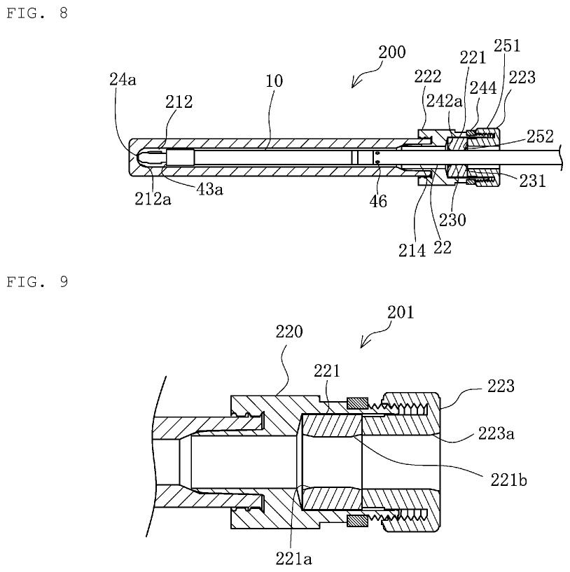

A modification of the priming instrument will be described. In a priming instrument 201 according to a first modification, as shown in FIG. 9, a tapered surface portion 221a and a tapered surface portion 221b are formed on a proximal portion and a distal portion of an inner peripheral surface of the seal member 221, respectively. Since the seal member 221 includes the tapered surface portions 221a and 221b as described above, the medical device 10 can be prevented from being caught by the seal member 221 when the medical device 10 is inserted into or removed from the hollow portion 212, and adhesion of fine particles to the medical device 10 due to breakage of the medical device 10 or the seal member 221 can be prevented. In addition, a tapered surface portion 223a is also formed on a proximal portion of an inner peripheral surface of the cap member 223. Accordingly, it is possible to facilitate the insertion of the medical device 10 by preventing the distal portion of the medical device 10 from being caught at the time of the insertion of the medical device 10.

As shown in FIG. 10, a priming instrument 202 according to a second modification includes a filter portion (or filter) 216 at a distal portion of the instrument body 210. The filter portion 216 allows air and a small amount of fluid to pass through the filter portion 216. As such a filter portion 216, for example, an acrylic copolymer, a polyether sulfone film (PES), nylon, acrylic (PMMA), polypropylene (PP), or polyethylene terephthalate (PTFE) can be used. The filter portion 216 allows a fluid to flow while discharging the air bubbles generated in the hollow portion 212 during priming to an outside. Therefore, the air bubbles can be prevented from remaining in the medical device 10.

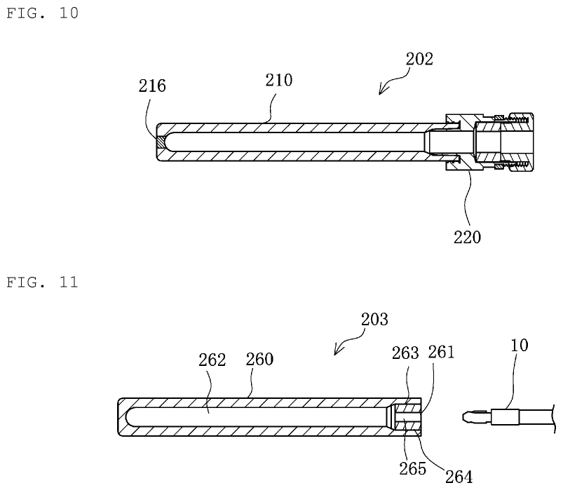

As shown in FIG. 11, a priming instrument 203 according to a third modification includes an instrument body 260 having an insertion port 261 and a hollow portion 262, and a sealing portion 263 provided on the insertion port 261. The sealing portion 263 includes a seal member 264 having an inner diameter smaller than the outer diameter of the medical device 10. The seal member 264 is formed of a flexible and elastic material, and a seal lumen 265 can be expanded in a radial direction. By inserting the medical device 10 into the seal lumen 265, the seal lumen 265 can be expanded in the diameter to allow the medical device 10 to be inserted.

As shown in FIG. 12, when the medical device 10 is inserted into the hollow portion 262, the seal lumen 265 is brought into pressure contact with the medical device 10 and seals an inside of the hollow portion 262. In this way, the hollow portion 262 may be sealed by elasticity of the seal member 264 itself. It is noted that in the priming instrument 203 according to the third modification, the seal lumen 265 of seal member 264 may be formed in a cross-cut slit shape.

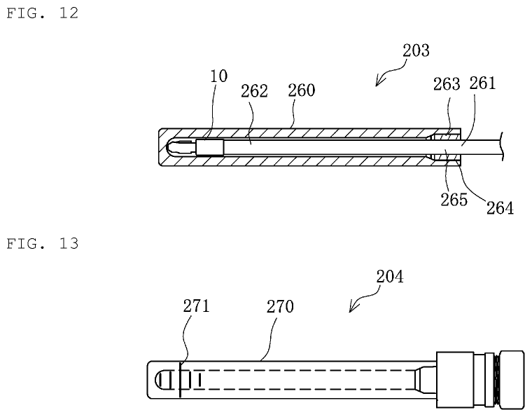

As shown in FIG. 13, in a priming instrument 204 according to a fourth modification, a marker portion 271 in which a plurality of lines are displayed is formed on an outer surface of an instrument body 270. Although the instrument body 270 is transparent or translucent, it may be difficult to know to which position the medical device 10 should be inserted when the medical device 10 is inserted. When the medical device 10 is excessively inserted, the distal portion may be damaged, and when the medical device 10 is insufficiently inserted, the opening portions may not be sufficiently covered by the hollow portion 212, and a liquid may be insufficiently delivered. Since the instrument body 270 includes the marker portion 271, the operator can rather easily insert the medical device 10 to an appropriate position.

Next, a method of using the medical device 10 according to the present embodiment will be described. Here, a case where a calcified lesion area in a blood vessel is destroyed and aspirated will be described as an example.

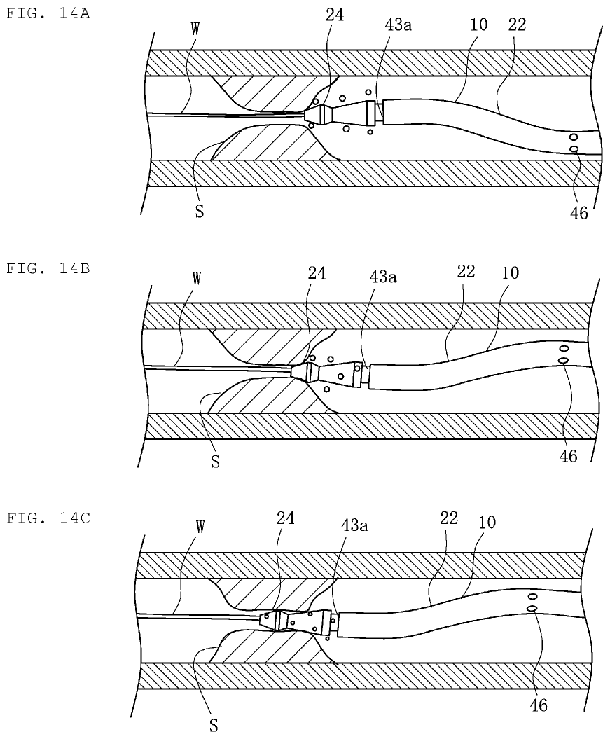

In advance, the operator performs priming of the medical device 10 using the above-described priming instrument 200. The operator inserts the guide wire W into the blood vessel and causes the guide wire W to reach the vicinity of a lesion area S. Next, the operator inserts a proximal end of the guide wire W into the guide wire lumen 27 of the medical device 10. Thereafter, as shown in FIG. 14A, the cutting portion 24 of the medical device 10 is moved to the vicinity of the lesion area S using the guide wire W as a guide.

Next, the operator operates the switch 53 to start operations of the drive unit 51 and the liquid delivering unit 52. Accordingly, the power shaft 51a of the drive unit 51 rotates, and the drive shaft 20 fixed to the power shaft 51a and the cutting portion 24 fixed to the drive shaft 20 rotate. Accordingly, the operator can cut the lesion area S by the cutting portion 24. When the power shaft 51a rotates, the conveying coil 32 disposed on the outer peripheral surface of the drive shaft 20 generates a force for conveying the liquid or the object in the discharge lumen 47 to the proximal side. An aspiration force is applied to the distal opening portion 43a of the outer tube shaft 22.

When the operator wants to change the position of the cutting portion 24 in a circumferential direction, the operator can operate the operation portion 54. When the operator rotates the operation dial 54a, the outer tube shaft 22 fixed to the operation portion 54 rotates. The seal holding portion 80 of the outer tube shaft 22 to which the operation portion 54 is fixed rotates inside the first liquid delivering lumen 64 of the housing 60. At this time, the first elastic bodies 90 and the second elastic bodies 91 slide on the inner peripheral surface of the first liquid delivering lumen 64. When the outer tube shaft 22 rotates, a direction of the distal portion of the shaping shaft 42 provided at the distal portion of the outer tube shaft 22 changes, and the position of the cutting portion 24 can be changed as shown in FIG. 14B. Therefore, cutting can be performed while changing the direction of the cutting portion 24 without rotating the entire handle portion 28 that is difficult to rotate greatly. Further, the operator moves the outer tube shaft 22 exposed to the entire handle portion 28 or the outside of the handle portion 28 in the axial direction to reciprocate the outer tube shaft 22 along a longitudinal direction of the blood vessel. Accordingly, as shown in FIG. 14C, the lesion area S can be cut along the longitudinal direction of the blood vessel by the cutting portion 24.

Since the flow of the fluid during a period from the operation of the liquid delivering unit 52 to the inflow of the saline into the liquid delivering lumen 45 is the same as that during the priming, the description of the flow of the fluid during a period from the operation of the liquid delivering unit 52 to the inflow of the saline into the liquid delivering lumen 45 will be omitted. At this time, the first elastic bodies 90 prevent the liquid in the first liquid delivering lumen 64 from leaking to the outside of the housing 60. In addition, the second elastic bodies 91 prevent the liquid in the second liquid delivering lumen 65 from leaking to the discharge lumen 66. In addition, the second sealing portion 41b that fixes the inner layer 41 and the seal holding portion 80 prevents the liquid flowing into the liquid delivering lumen 45 from leaking to the discharge lumen 66. Therefore, the saline that flowed into the first liquid delivering lumen 64 from the first liquid delivering port 62 is effectively guided to the liquid delivering lumen 45 while maintaining a relatively high liquid delivering pressure.

The saline discharged to the second liquid delivering tube 101 flows into the second liquid delivering lumen 65 of the housing 60 from the second liquid delivering port 63. The third elastic body 75 help prevent the liquid in the second liquid delivering lumen 65 from leaking to the outside of the housing 60. In addition, the saline supplied from the second liquid delivering tube 101 to the second liquid delivering lumen 65 flows into the discharge lumen 66 on the distal side.

The saline entering the liquid delivering lumen 45 is released from the liquid delivering opening portion 46 into the blood vessel. A part of the saline discharged into the blood vessel is, together with blood and the cut object, aspirated into the discharge lumen 47 from the distal opening portion 43a of the outer tube shaft 22. The object and the liquid entering the discharge lumen 47 move in the discharge lumen 47 toward the proximal direction. The object and the blood to be aspirated into the discharge lumen 47 are diluted with the saline. Therefore, a discharge amount is increased by decreasing viscosity of a discharged material, and formation of a thrombus in the discharge lumen 47 is prevented. Therefore, aspiration performance can be improved while preventing a decrease of the aspiration force or the damage of the medical device 10 due to the formation of the thrombus in the discharge lumen 47. In addition, the thrombus formed in the medical device 10 is prevented from flowing into the body lumen.

When the liquid or the object entering the discharge lumen 47 moves in the discharge lumen 47 toward the proximal direction, the liquid or the object reaches the discharge lumen 66 of the housing 60 from the proximal opening portion 48a on the inner layer 41. When the liquid or the object reaches the discharge lumen 66, the liquid or the object is discharged from the discharge port 61 to the external waste liquid bag via the discharge tube 103.

The saline flows into the discharge lumen 66 from the second liquid delivering lumen 65 on the proximal side. Accordingly, the liquid or the object flowing from the discharge lumen 47 into the discharge lumen 66 is diluted by the saline. Therefore, the discharge amount is increased by decreasing the viscosity of the discharged material, and formation of thrombi in the discharge lumen 47, the discharge lumen 66, and the discharge tube 103 is prevented. Therefore, the aspiration performance can be improved while preventing the decrease of the aspiration force or the damage of the medical device 10 due to the formation of the thrombi in the discharge lumen 47, the discharge lumen 66, and the discharge tube 103.

In addition, the second liquid delivering port 63 is disposed adjacent to the shaft support 70 located in the second liquid delivering lumen 65. Therefore, the shaft support 70 can be cooled and lubricated by the saline from the second liquid delivering port 63.

After the cutting and the aspiration of the lesion area S are completed, the operator presses the switch 53. Accordingly, the rotation of the drive shaft 20 is stopped, and the liquid delivery performed by the liquid delivering unit 52 is stopped. Thereafter, the operator removes the medical device 10 from the blood vessel and completes the procedure.

As described above, the priming instrument 200 for the medical device 10 according to the present embodiment is the priming instrument 200 for the medical device 10 including the liquid delivering lumen 45 having the first opening portion 46 and the discharge lumen 47 having the second opening portion 43a. The priming instrument 200 includes: the instrument body 210 having the hollow portion 212 that covers the first opening portion 46 and the second opening portion 43a of the medical device 10, in which the hollow portion 212 includes the distal portion 212a and the proximal portion 212b, the distal portion 212a of the hollow portion 212 is closed, the proximal portion 212b of the hollow portion 212 has the insertion port 214 into which the medical device 10 is insertable, and the insertion port 214 includes the sealing portion 220 that seals the inside of the hollow portion 212 in the state in which the medical device 10 is inserted into the hollow portion 212. In the medical device 10 configured as described above, each of the lumens 26, 47, and 45 can be filled with the fluid through the sealed hollow portion 212. At this time, since a rotating portion of the medical device 10 is not required to be rotated and the aspiration is not required, the priming can be rather easily performed. In addition, the generation of the air bubbles due to the rotation of the rotating portion can be prevented.

In addition, the filter portion 216 that allows air and a small amount of fluid to pass through the filter portion 216 may be provided at the distal portion 212a of the hollow portion 212. Accordingly, it is possible to cause the fluid to flow while discharging the air bubbles generated in the hollow portion 212, and thus it is possible to help prevent the air bubbles from remaining in the medical device 10.

In addition, the distal portion 212a of the hollow portion 212 facing the distal portion of the medical device 10 may have the concave curved surface shape. Accordingly, the distal portion of the medical device 10 can be prevented from being damaged, and the distal end of the medical device 10 can be prevented from being sealed, so that it is possible to cause the fluid to reliably flow into the lumens.

In addition, the instrument body 210 may be formed of the flexible material at the distal portion where the distal portion of the medical device 10 is disposed, or the entire instrument body 210 may be formed of the flexible material. Accordingly, the medical device 10 inserted into the instrument body 210 is prevented from being damaged.

In addition, the sealing portion 220 may include the seal member 221 having the lumen 230 of which the inner diameter is larger than the outer diameter of the medical device 10 and is reduced by the compression in the axial direction, and the compression surface portion 252 that is movable along the axial direction with respect to the seal member 221 and compresses the seal member 221 in the axial direction. Accordingly, the operator can reliably compress the seal member 221 and seal the hollow portion 212 by performing an operation to move the compression surface portion 252.

In addition, the sealing portion 220 may include the stopper portion 244 that restricts the compression surface portion 252 from moving by a predetermined distance or more in the axial direction. Accordingly, the compression surface portion 252 can prevent the seal member 221 from being excessively compressed and prevent the medical device 10 sealed with the seal member 221 from being damaged.

In addition, the sealing portion 220 may include the flexible seal member 264 that has the inner diameter smaller than the outer diameter of the medical device 10 and is expandable in the radial direction. Accordingly, the hollow portion 262 into which the medical device 10 is inserted can be sealed only by inserting the medical device 10 into the seal member 264.

In addition, the medical device system according to the present embodiment includes the medical device 10 that has the plurality of lumens 26, 47, and 45 having the opening portions 24a, 43a, and 46, respectively, and the priming instrument 200 according to the present embodiment. The medical device system configured as described above can rather easily and reliably perform the priming of the medical device.

In addition, a method for priming the medical device 10 according to the present embodiment is a method for priming the medical device 10 including the liquid delivering lumen 45 having the first opening portion 46 and the discharge lumen 47 having the second opening portion 43a. The method includes: inserting the distal portion of the medical device 10 from the insertion port 214 of the instrument body 210 and disposing the first opening portion 46 and the second opening portion 43a of the medical device 10 in the hollow portion 212 of the instrument body 210; sealing the inside of the hollow portion 212 with the sealing portion 220 provided in the insertion port 214 of the instrument body 210; and injecting the fluid into the liquid delivering lumen 45 from the proximal side, causing the fluid to flow into the hollow portion 212 from the first opening portion 46 of the liquid delivering lumen 45 into which the fluid is injected, and causing the fluid to flow into the discharge lumen 47 from the hollow portion 212 via the second opening portion 43a, whereby the liquid delivering lumen 45 and the discharge lumen 47 are filled with the fluid. The method for priming the medical device 10 configured as described above can rather easily perform the priming while preventing the damage of the medical device 10 and the generation of the air bubbles.

In addition, in the method for priming the medical device 10 according to the present embodiment, the medical device 10 further includes the guide wire lumen 27 having the third opening portion 24a, the first opening portion 46, the second opening portion 43a, and the third opening portion 24a of the medical device 10 are disposed in the hollow portion 212 by inserting the distal portion of the medical device 10 into the instrument body 210, the fluid is caused to flow from the first opening portion 46 of the liquid delivering lumen 45 into the hollow portion 212, the fluid is caused to flow into the discharge lumen 47 from the hollow portion 212 via the second opening portion 43a, and the fluid is caused to flow into the guide wire lumen 27 from the hollow portion 212 via the third opening portion 24a, whereby the liquid delivering lumen 45, the discharge lumen 47, and the guide wire lumen 27 are filled with the fluid. Accordingly, the priming including priming of the guide wire lumen 27 can be easily performed.

The present disclosure is not limited to the embodiments described above, and various modifications can be made by those skilled in the art within a scope of the technical idea of the present disclosure. Although the above-described embodiments have three lumens filled with the liquid by the priming, the priming instrument 200 according to the present embodiment can be used as long as a medical device has two or more lumens. In addition, the type of the medical device is not limited to an atherectomy device, and other types of medical devices may be used.

The detailed description above describes embodiments of a priming instrument for a medical device having a plurality of lumens that open in a living body, a medical device system, and a method for priming a medical device. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A priming instrument for a medical device including a liquid delivering lumen having a first opening portion and a discharge lumen having a second opening portion, the priming instrument comprising:
an instrument body having a hollow portion configured to cover the first opening portion and the second opening portion of the medical device;
the hollow portion including a distal portion and a proximal portion, the distal portion of the hollow portion being closed, and the proximal portion of the hollow portion having an insertion port into which the medical device is insertable; and
the insertion port includes a sealing portion configured to seal an inside of the hollow portion in a state where the medical device is inserted into the hollow portion.

2. The priming instrument according to claim 1, further comprising:
a filter provided at the distal portion of the hollow portion, the filter configured to allow air and fluid to pass through the filter.

3. The priming instrument according to claim 1, wherein the distal portion of the hollow portion facing a distal portion of the medical device has a concave curved surface shape.

4. The priming instrument according to claim 1, wherein the instrument body is formed of a flexible material at a distal portion where a distal portion of the medical device is disposed.

5. The priming instrument according to claim 1, wherein the instrument body is entirely formed of a flexible material.

6. The priming instrument according to claim 1, wherein the sealing portion includes a seal member having a lumen of which an inner diameter of the lumen of the sealing member is larger than an outer diameter of the medical device and the sealing member is configured to be reduced by compression in an axial direction, and a compression surface portion that is movable along the axial direction with respect to the seal member and configured to compress the seal member in the axial direction.

7. The priming instrument according to claim 6, wherein the sealing portion includes a stopper portion configured to restrict the compression surface portion from moving by a predetermined distance or more in the axial direction.

8. The priming instrument according to claim 1, wherein the sealing portion includes a flexible seal member that has an inner diameter smaller than an outer diameter of the medical device and is expandable in a radial direction.

9. A medical device system comprising:
a medical device that includes a liquid delivering lumen having a first opening portion and a discharge lumen having a second opening portion; and
a priming instrument, the priming instrument comprising:
an instrument body having a hollow portion that covers the first opening portion and the second opening portion of the medical device;
the hollow portion including a distal portion and a proximal portion, the distal portion of the hollow portion being closed, and the proximal portion of the hollow portion has an insertion port into which the medical device is insertable; and
the insertion port includes a sealing portion configured to seal an inside of the hollow portion in a state where the medical device is inserted into the hollow portion.

10. The medical device system according to claim 9, wherein the priming instrument includes a filter provided at the distal portion of the hollow portion, the filter configured to allow air and fluid to pass through the filter.

11. The medical device system according to claim 9, wherein the distal portion of the hollow portion of the priming instrument facing a distal portion of the medical device has a concave curved surface shape.

12. The medical device system according to claim 9, wherein the instrument body of the priming instrument is formed of a flexible material at a distal portion where a distal portion of the medical device is disposed.

13. The medical device system according to claim 9, wherein the instrument body of the priming instrument is entirely formed of a flexible material.

14. The medical device system according to claim 9, wherein the sealing portion of the priming instrument includes a seal member having a lumen of which an inner diameter of the lumen of the sealing member is larger than an outer diameter of the medical device and the sealing member is configured to be reduced by compression in an axial direction, and a compression surface portion that is movable along the axial direction with respect to the seal member and configured to compress the seal member in the axial direction.

15. The medical device system according to claim 14, wherein the sealing portion of the priming instrument includes a stopper portion configured to restrict the compression surface portion from moving by a predetermined distance or more in the axial direction.

16. The medical device system according to claim 14, wherein the sealing portion of the priming instrument includes a flexible seal member that has an inner diameter smaller than an outer diameter of the medical device and is expandable in a radial direction.

17. A method for priming a medical device including a liquid delivering lumen having a first opening portion and a discharge lumen having a second opening portion, the method comprising:
inserting a distal portion of the medical device from an insertion port of an instrument body and disposing the first opening portion and the second opening portion of the medical device in a hollow portion of the instrument body;
sealing an inside of the hollow portion with a sealing portion provided in the insertion port of the instrument body; and
injecting a fluid into the liquid delivering lumen from a proximal side, causing the fluid to flow into the hollow portion from the first opening portion of the liquid delivering lumen into which the fluid is injected, and causing the fluid to flow into the discharge lumen from the hollow portion via the second opening portion to fill the liquid delivering lumen and the discharge lumen with the fluid.

18. The method according to claim 17, wherein the medical device further includes a guide wire lumen having a third opening portion, further comprising:
inserting the distal portion of the medical device into the instrument body to dispose the first opening portion, the second opening portion, and the third opening portion of the medical device in the hollow portion; and
causing the fluid to flow into the hollow portion from the first opening portion of the liquid delivering lumen, causing the fluid to flow into the discharge lumen from the hollow portion via the second opening portion, and causing the fluid to flow into the guide wire lumen from the hollow portion via the third opening portion so as to fill the liquid delivering lumen, the discharge lumen, and the guide wire lumen with the fluid.

19. The method according to claim 17, further comprising:
providing a filter at the distal portion of the hollow portion; and
allowing air and fluid to pass through the filter.

20. The method according to claim 17, wherein the distal portion of the hollow portion of the priming instrument facing a distal portion of the medical device has a concave curved surface shape.
